(19) United States
(12) Patent Application Publication
(10) Pub. No.: US 2005/0148653 A1
(43) Pub. Date: Jul. 7, 2005

(54) Polymaleimide compounds and their medical use

(76) Inventors: Suren Solanki, Cambridge (GB); William Duncan Potter, Cambridge (GB); Lorraine Anderson, Edinburgh (GB)

Correspondence Address: STERNE, KESSLER, GOLDSTEIN & FOX PLLC, 1100 NEW YORK AVENUE, N.W., WASHINGTON, DC, 20005, US

(21) Appl. No.: 10/493,428

(22) PCT Filed: Oct. 24, 2002
(86) PCT No.: PCT/GB02/04798

(30) Foreign Application Priority Data
Oct. 25, 2001 (GB) 0125658.5

(51) Int. Cl. A61K 31/4025; C07D 43/02

(52) U.S. Cl. 514/422; 548/519

(57) ABSTRACT

The present invention relates to the use of poly-maleimide derivatives as medicaments. In particular it relates to the use of a maleimide compound of formula (I) or a salt, ester or other physiologically equivalent derivative thereof for use in the manufacture of a medicament for the prophylaxis and/or treatment of a bacterial, viral, fungal or protozoal infection of the reproductive system. The invention further provides such compounds and their use in medicine.

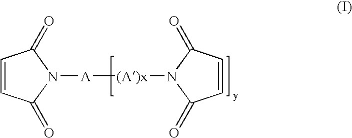

The present invention relates to the use of poly-maleimide derivatives as microbicidal agents. In particular it relates to the use of poly-maleimide derivatives, preferably bis-maleimide compounds in the treatment of microbial infections of the reproductive tract, such as bacterial, fungal, protozoal and viral infections.

Despite efforts to reduce the levels of sexually transmitted infections (STIS) for example by providing relevant information and education, and by promoting the use of barrier contraceptives, such as condoms, the level of such infections worldwide is increasing. There is therefore a continuing requirement for safe and effective therapeutic agents for the prophylaxis and treatment of STI's, in particular, agents which can readily be administered or applied at the time of sexual contact. Maleimides are a well known class of chemical compounds.

Maleimide derivatives are known to have a wide variety of utilities, and find application in the biological and medicinal fields, as well as in general industrial use, for example in the preparation of polymers and inks.

Thus for example, U.S. Pat. No. 3,129,225 (United States Vitamin and Pharmaceutical Corporation) describes maleimido betaines said to have antimicrobial and antiparasitic activity as well as pharmacological activity, in particular the reduction of serum cholesterol.

German ALS 1,140,192 (United States Rubber Company) describes a novel bismaleimide with a CH₂—O—CH₂ bridge, and its use in vulcanisation, cross-linking of polymers and as fungicide for tomatoes.

U.S. Pat. No. 4,876,358 (Texaco Inc.) describes novel bismaleimides having a (poly)oxyethylene bridge, for use in preparing polymers.

U.S. Pat. No. 5,552,396 and WO 97/19080 (Eli Lilly) describe classes of N,N-bridged bisindolylmaleimides which are said to be inhibitors of protein kinase C, and to be useful for the treatment of diabetes, ischemia, inflammation, cns disorders, cardiovascular disease, dermatological disease and cancer.

U.S. Pat. No. 5,380,764 (Goedecke AG) describes bis-indolemaleinimides,said to be protein kinase C inhibitors useful for treating diseases of heart or blood vessels, inflammatory processes, allergies, cancer and degenerative damage of cns, diseases of the immune system and viral diseases, including diseases caused by retroviruses HTLV-I, -II, -III.

WO 96/09406 (The Government of the United States of America, represented by the Secretary of the Department of Health and Human Services) describes the use of disulfides, maleimides and a variety of other electron acceptor compounds in inactivating FHV-1 and other retroviruses.

Igarashi et al., J. Industrial Microbiology, 6 (1990) pp223-225, report that various N-alkyhnaleimides show antibacterial and antifungal activity.

We have found that poly-maleimide derivatives inhibit the infectivity of the human HV-1 virus. Such maleimides of formula (1) are useful in the prophylaxis and/or treatment of a variety of sexually transmitted infections and other infections which affect the reproductive system, including reproductive organs and reproductive tract.

In a first aspect, therefore, the present invention provides the use of a poly-maleimide derivative in the manufacture of a medicament for the prophylaxis and/or treatment of microbial infections, in particular, sexually transmitted infections and other infections which affect the reproductive system.

Microbial STIs and other infections which affect the reproductive system include bacterial, fungal, protozoal and viral infections.

Bacterial infections include, chancroid, chlamydia, gonorrhoea, syphylis and bacterial vaginosis. Causitive agents of such infections include H. ducreyi, C. trachomatis, N. gonorrhoea, T. pallidum and anaerobic bacteria such as G. vaginalis.

Fungal infections include candidiasis (commonly known as thrush) which is caused by C. albicans.

Protozoal infections include trichomoniasis, caused by T. vaginalis.

Viral STI's include those caused by a virus selected from IRV (AIDS) HSV (herpes simplex) HPV(human papilloma virus) and HBV (hepatitis B).

The present invention therefore provides the use of a poly-maleimide compound in the manufacture of a medicament for the prophylaxis and/or treatment of a bacterial, fungal or protozoal infection of the reproductive system. The term poly-maleimide compound or derivative includes all derivatives, salts and substituted maleimides, including optionally substituted bis-maleimides and poly-maleimides. It does not relate to mono-maleimide compounds.

The present invention also provides the use of a maleimide derivative in the manufacture of a medicament for the prophylaxis and/or treatment of a viral infection.

The maleimide compound may preferably be represented by the general formula (I): 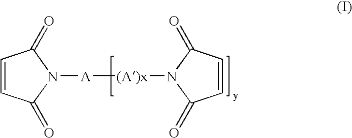
wherein
A is represented by (R¹)ₙ—(R²)ₚ—(R³)_{q}, wherein each of R¹, R² and R³ is independently selected from:
an acyclic aliphatic group having from 1-20 carbon atoms;
a polyalkylene glycol group having an average molecular weight in the range 100 to 1000;
a cyclic aliphatic group having from 4-20 carbon atoms;
a monocyclic or polycyclic aromatic hydrocarbon group having from 6-18 ring carbon atoms;
a non-aromatic heterocyclic group having from 3 to 8 ring atoms, or
a monocyclic or polycyclic aromatic heterocyclic group having from 5 to 20 ring atoms;
and n, p and q each represent 0 or 1 such that the sum of n+p+q=1-3;
or A substituted by one or more groups independently selected from: 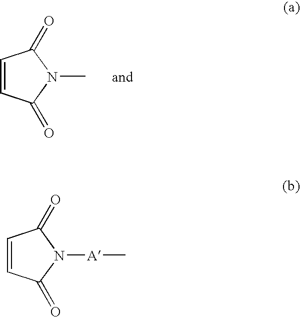
wherein A′, which may be the same as or different from A, represents a group (R¹)ₙ—(R²)ₚ—(R³)_{q}, wherein R¹ , R² , R³, n, p and q are as defined above and wherein X is 0 or 1 and y is 1 to 6;
   or a salt, ester or other physiologically equivalent derivative thereof.

In compounds of formula (I) it is preferred that the total number of atoms in the moieties A and A′ does not exceed 50.

In the compounds of formula (I) above, an acyclic aliphatic group or moiety may be branched or unbranched alkyl, which alkyl group may be optionally interrupted by one or more oxygen atoms; alkenyl; or alkynyl; any of which may be optionally substituted by one or more substituents selected from the group consisting of halogen, cyano, nitro, —C(O)R⁴, —CO₂R⁴, —OR⁴, —SR⁴—SOR⁴, —SO₂R⁴, —NR⁴R⁵, —C(O)NR⁴R⁵; wherein R⁴ and R⁵ independently represent hydrogen, C₁₋₈alkyl, aryl or arylC₁₋₈alkyl.

For the purposes of the present invention, alkyl groups may have from 1 to 20 carbon atoms, preferably from 1 to 15 carbon atoms, more preferably from 1 to 10 carbon atoms. Alkenyl groups may have from 2 to 20 carbon atoms, preferably from 2 to 15 carbon atoms, more preferably from 2 to 10 carbon atoms. Alkynyl groups may have from 2 to 20 carbon atoms, preferably from 2 to 15 carbon atoms, more preferably from 2 to 10 carbon atoms.

A polyalkyleneglycol group may be a polyethylene glycol (PEG) or a polypropylene glycol which may be represented by the formulae —(OCH₂CH₂)ₘOH, —(OCH(CH₃)CH₂)ₘOH or —(OCH₂CH₂CH₂)ₘOH, wherein m is in the range 4 to 20. It will be appreciated that polyalkylene glycols are generally a mixture of chain lengths and are therefore conventionally described in terms of average molecular weight and average values of m. Thus for example PEG 200 has molecular weight in the range 190-210 and an average value of m=4 (The Merck Index, Eleventh edition).

Aryl is phenyl optionally substituted by one or more substituents selected from the group consisting of halogen, cyano, nitro, —C(O)R⁶, —CO₂R⁶, —OR⁶, —SR⁶ SOR⁶, —SO₂R⁶, —NR⁶R⁷, —C(O)NR⁶R⁷ wherein R⁶ and R⁷ independently represent hydrogen, or C₁₋₈alkyl.

A cyclic aliphatic group or moiety may be cycloalkyl or cycloalkenyl, either of which may be optionally substituted by one or more substituents selected from the group consisting of halogen, cyano, nitro, —C(O)R⁴, —CO₂R⁴, —OR⁴, —SR⁴—SOR⁴, —SO₂R⁴, —NR R⁵, —C(O)NR⁴R⁵; wherein R⁴ and R⁵ are as defined above.

Cycloalkyl groups of the present invention may have from 3 to 20 carbon atoms, preferably from 3 to 10 carbon atoms, more preferably from 3 to 6 carbon atoms. Cycloalkenyl groups of the present invention may have from 3 to 20 carbon atoms, preferably from 3 to 10 carbon atoms, more preferably from 3 to 6 carbon atoms.

Aromatic hydrocarbon groups and moieties include for example optionally substituted unsaturated monocyclic, fused bicyclic or tricyclic rings of up to 18 carbon atoms, such as phenyl and naphthyl, and partially saturated bicyclic or tricyclic rings such as tetrahydro-naphthyl. Aromatic groups also include linked aromatic rings, such as a biphenyl group. The aromatic rings may also be linked by heteroatoms, e.g. O, S or NR⁴. Examples of substituents which may be present on an aromatic group or moiety include one or more of halogen, alkyl, haloalkyl, cyano, nitro, —C(O)R⁴, —CO₂R⁴, —OR⁴, —SR⁴—SOR⁴, —SO₂R⁴, —NR⁴R⁵, or —C(O)NR⁴R⁵. Substituents on the aromatic ring are preferably in the ortho or para position with respect to the maleimido group.

An aromatic heterocyclic group or moiety may be unsaturated or partially saturated, for example an optionally substituted 5- or 6-membered heterocyclic aromatic ring which may contain from 1 to 4 heteroatoms selected from O, N and S. The heterocyclic ring may optionally be fused to one or more phenyl rings. Examples of heteroaryl groups thus include furyl, thienyl, pyrrolyl, oxazolyl, oxazinyl, thiazolyl, imidazolyl, oxadiazolyl, thiadiazolyl, pyridyl, triazolyl, triazinyl, pyridazyl, pyrimridinyl, pyrazolyl, indolyl, indazolyl, benzofuranyl, benzothienyl, benzimidazolyl, benzoxazolyl, benzoxazinyl, quinoxalinyl, quinolinyl, quinazolinyl, cinnolinyl, benzothiazolyl, pyridopyrrolyl. Examples of substituents which may be present on a heterocyclic aromatic group include one or more of halogen, oxo,, nitro, cyano, alkyl, haloalkyl, —C(O)R⁴, —CO₂R⁴, —OR⁴, —SR⁴—SOR⁴, —SO₂R⁴, —NR⁴R⁵, or —C(O)NR⁴R⁵, wherein R⁴ and R⁵ are as defined above. Partially saturated derivatives of the aforementioned heteroaryl groups include 3-pyrroline and 1,2-dihydroquinoline.

Non-aromatic heterocyclic groups include pyrrolidinyl, tetrahydrofuryl, tetrahydrothienyl and piperidinyl.

In the compounds of formula (I) R¹ preferably represents an aromatic hydrocarbon group, e.g. phenyl or naphthyl; or an aromatic heterocyclic group e.g. thienyl or acridine. Optional substituents present on the phenyl group are preferably in the ortho or para position (relative to maleimido) and may be for example selected from amino, halogen, eg chlorine, C₁₋₄alkyl, nitro, phenoxy and phenylamino.

When A represents phenyl the maleimido moieties are preferably in the para position relative to each other. When A represents biphenyl the bonds between the maleimido and phenyl groups and the bond between the two phenyl groups are are also preferably in the para positions.

A preferred class of compounds for use according to the invention is that of bismaleimide derivatives represented by formula (II): 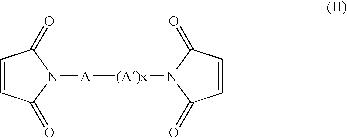
wherein A, A¹ and x are as defined above.

In the compounds of formula (II) x preferably represents 0 and A is as defined above.

In the group A, R¹, R², R³, n, p and q are preferably selected such that A represents a C₁₋₈alkyl group, optionally interrupted by an oxygen atom;
a polyalkylene glycol group having an average molecular weight in the range 100 to 1000; or
a phenyl group; or
n and q are preferably each 1, p represents 0 or 1,R¹ and R³ preferably each represent phenyl and, when p is 1, R² preferably represents a C₁₋₈alkyl group, optionally interrupted by an oxygen atom; or
a polyalkylene glycol group having an average molecular weight in the range 100 to 1000.

When A represents phenyl, the maleimido moieties are preferably in the para position relative to each other. When A represents biphenyl, the bonds between the maleimido and phenyl groups and the bond between the two phenyl groups are are also preferably in the para positions.

Maleimide derivatives for use according to the present invention are commercially available or can be prepared according to methods well known in the art.

A maleimide derivative for use according to the present invention may be administered as the sole active ingredient or may be administered in combination with one or more other maleimide derivatives as defined herein and/or in combination with one or more other therapeutic agents. In this context in combination means that the compounds may be administered simultaneously or sequentially, and there may be an interval of time between administration of the two or more compounds. Other therapeutic agents which may be administered in combination with one or more maleimide derivatives include antibacterial, antifungal and antiviral agents.

In a second aspect, the present invention provides the use of a compound of formula (I) and preferably the use of a compound of formula (II) for the prophylaxis and/or treatment of retroviral infections of the reproductive tract, in particular, HIV infection.

The third aspect of the invention provides a pharmaceutical formulation comprising a compound as defined for the first aspect and a pharmaceutically acceptable excipient.

For use in the treatment of STIs the compounds may be formulated for administration to the reproductive organs or reproductive tract (vaginal, cervical or uterine) or for rectal, buccal or oral administration. For administration to the reproductive organs or reproductive tract, or rectal administration the compounds may be formulated using conventional methods known in the art. Thus for example they may be formulated as creams, gels including thermoreversible gels, lubricants, pessaries, foaming tablets, aerosol foams, sprays, douches or films. The compounds may be incorporated into sustained release formulations, e.g. by micro-encapsulation, formulation with a bio-adhesive material, such as poly-carbophil, or other conventional means. Gels, creams and pessaries may, according to circumstance, be administered via an appropriate applicator. The formulations, in particular gels, creams and pessaries may be packaged with an appropriate applicator to facilitate administration.

Compounds and compositions for use according to the present invention may also be incorporated into contraceptive devices. Thus for example they may be incorporated into vaginal, cervical or uterine devices such as vaginal sponges or rings, and diaphragms or used to lubricate and/or coat condoms.

For buccal and oral administration the compounds may be formulated as lozenges, chewable tablets, wafers, mouthwashes and the like.

For use according to the present invention, a maleimide derivative may be administered at a dosage in the range 10-1000 mg, e.g. 30 to 1000 mg.

The fourth aspect of the invention provides a method of treating a microbial infection which affects the reproductive system comprising administering to a subject in need thereof a compound as defined in the first aspect or a composition or device as defined in the third aspect.

A fifth aspect of the invention provides a compound of formula (V) 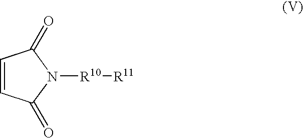
wherein R¹¹ is 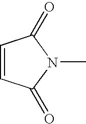
and R¹⁰ is C₂₋₁₀ alkyl, preferably wherein the alkyl group has 2, 3, 4, or 5 carbon atoms, or a group R²⁰-R²¹-R²² wherein R²⁰ is aryl, preferably phenyl;
R²¹ is —(OCH₂CH₂),— wherein in represents 3 to 15, preferably n is one of 4, 5, 6, 7, 8, 9, 10, 11,12 and 13;
R²² is a group O—R²³ wherein R²³ is aryl or C₁₋₁₀ alkyl, preferably phenyl or C₁₋₄ alkyl, more preferably phenyl or methyl.

Preferred compounds of the fifth aspect are set out in the table below.

| Compound | |
|---|---|
| number | Structure |
| 1 | 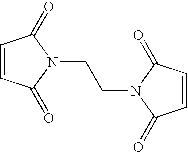 |
| 2 | 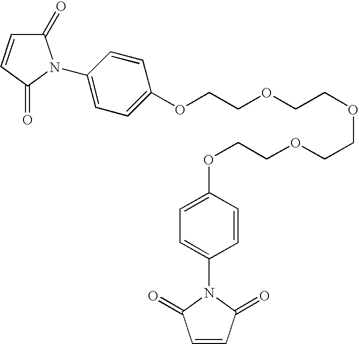 |

The fifth aspect of the invention also relates to salts, esters, amides or prodrugs of the compounds of formula V.

The compounds of the fifth aspect can be synthesised according to methods well known in the art. In particular, PEG of the appropriate chain length is initially converted to the aminoaryl derivative using conventional chemical synthesis. One example of such a chemical synthesis includes the coupling of PEG with p-fluoronitrobenzene in the presence of sodium hydride and DMF to produce a nitro-substituted aryl-PEG moiety. The nitro-substituted aryl-PEG moiety is then reduced with a reducing agent such as palladium/carbon to form the required amine compound. The amine compound can then be converted into the maleimide compound of formula V by a number of methods known in the art. One example of such a method includes reacting the amine with maleic anhydride.

The sixth aspect of the invention provides a compound of formula V as defined in the fifth aspect of the invention for use in medicine. The compound of formula V is preferably provided as for the prevention or treatment of microbial infections which effect the reproductive tract. The compounds can be used to prevent or treat sexually transmitted infections and other infections which affect the reproductive tract.

All preferred features of different aspects of the invention apply to each other mutatis mutanadis.

The invention will be further illustrated by the following examples:

Biological Testing

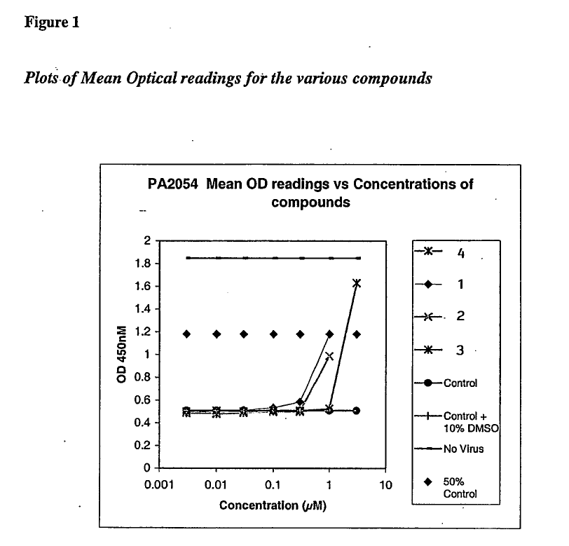

FIG. 1 indicate optical density measurements versus various concentrations of the compounds of the invention.

Methods

Chlamydia Assay

Purified Chlamydia trachomatis (250 μl) was added to 250 μl of each compound concentration and the mixtures incubated at room temperature for 5 minutes. The tubes were then centrifuged (15000 rpm, 15 minutes), the supernatant decanted and the pellet washed serum free MEM (with 0.5 mls). This step was repeated and the resultant pellet resuspended MEM (in 0.5 ml) and inoculated into a confluent shell vial (coverslip) cultures of McCoy cells. The cultures were centrifuged (3000 rpm, 1 hour at 37° C.) and incubated for a further 2 hours prior to being changed onto MEM with 10% foetal calf serum, I μg/rl cycloheximide, 0.5% added glucose, vancomycin, streptomycin and amphoteracin B (Fungizone). The cultures were incubated for 68 hours, fixed in methanol and stained with Lugol's iodine. 3 replicates of each positive control (with and without DMSO) were included, as well as 2 uninoculated cell controls. Each coverslip culture was examined using low power (10×) objective to ensure that inclusions were evenly distributed and 5 high power (×40) fields were selected at random from different areas of the coverslip. Iodine stained inclusions were counted. Where no inclusions were seen on high power, the culture was re-examined under low power.

Anti-Viral Assay

HIV was attached to the surface of 96-well cell culture plates using 100 μl of 50 μg/ml poly-L-lysine hydrobromide (PLL) of molecular weight >300 K for 1 h at RT. The outer wells were left uncoated and filled with 250 μl of PBS to counteract the effects of evaporation. The coated wells were washed twice with PBS and 25 μl of RPMI 1640 medium containing 5×10³ TCID₅₀ of IHV-1_{RF} was added and incubated for 1 h at RT. The wells were then washed twice with PBS and 25 μl of varying concentrations of the test compounds made up with serum free medium (containing a constant 10% DMSO) were added to the wells in quadruplicate and the plates incubated at 37° C. for 10 minutes. The wells were again washed twice with PBS and 5×10⁴ C8166 cells in a volume of 300 μl distributed to each well and the plates incubated for 5 and 6 days respectively. On days 5 and 6 the cells were assessed microscopically for the presence of cytopathic effect (syncytium formation) and recorded. In order to quantify the cytopathic effect, XIT which is metabolised by viable but not dead cells, was added and the absorbance read at 450 nm after 34 hr incubation at 37° C.

Test Compounds

The following compounds of the invention were tested as set out in the above methods.

| Compound | |
|---|---|
| number | Structure |
| 1 | 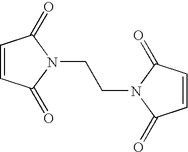 |
| 2 | 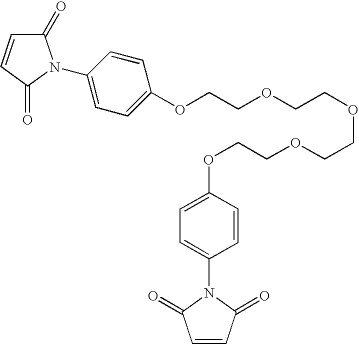 |

The comparative compounds, ethyl maleimide and PEG(5)PhMA were also used in the above methods.

| | |
|---|---|
| Comparative compound 3 (PEG(5)PhMA) | 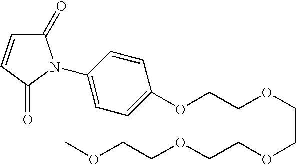 |
| Comparative compound 4 (Ethyl maleimide) | 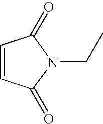 |

Results
Chlamydia Assay: Table 1

There is no apparent difference between the number of inclusions found in McCoy cells exposed to chlamydia transiently treated with either media (DMEM) or vehicle treated chlamydia (DMSO, 10% final) suggesting that this concentration of DMSO does not compromise the ability of chlamydia to infect McCoy cells. Transient exposure of chlamydia to compounds 4, 1, 3 and 2 resulted in a dose-dependent inhibition of the infection of McCoy cells with chlamydia. Ethyl maleimide (4) is less active than its bis maleimide equivalent, ethyl bis-maleimide (1). Similarly PEG(5) phenyl maleimide (3) is less active than its PEG bis (phenyl maleimide) equivalent, PEG(5) bis phenyl bis maleimide (2).

TABLE 1

| | | | | IODINE-STAINED INCLUSIONS/FIELD | | | | | | | |
|---|---|---|---|---|---|---|---|---|---|---|---|
| | | WORKING | FINAL | | | | | | TOTAL | MEAN | |
| | | CONC | CONC | | | | | | COUNT/ | COUNT/ | |
| TREATMENT | REPLICATE | (M) | (M) | F1 | F2 | F3 | F4 | F5 | FIELDS | FIELD | COMMENTS |
| Uninoculated | | | | 0 | 0 | 0 | 0 | 0 | 0 | 0 | No |
| Cell | | | | | | | | | | | inclusions |
| Control | | | | | | | | | | | seen |
| Media | | | | 70 | 50 | 68 | 58 | 63 | 309 | 62 | |
| (DMEM) | | | | | | | | | | | |
| Chlamydia | | | | | | | | | | | |
| Inoculated | | | | | | | | | | | |
| Cells | | | | | | | | | | | |
| Vehicle | | | | 75 | 71 | 61 | 84 | 75 | 366 | 73 | |
| Control | | | | | | | | | | | |
| Chlamydia | | | | | | | | | | | |
| Inoculated | | | | | | | | | | | |
| Cells | | | | | | | | | | | |
| (10% | | | | | | | | | | | |
| DMSO) | | | | | | | | | | | |
| 4 | 1 | 2 | 1 | 0 | 0 | 0 | 0 | 0 | 0 | 0 | No |
| | | | | | | | | | | | inclusions |
| | | | | | | | | | | | seen |
| | 2 | 0.8 | 0.4 | 0 | 0 | 0 | 0 | 0 | 0 | 0 | No |
| | | | | | | | | | | | inclusions |
| | | | | | | | | | | | seen |
| | 3 | 0.04 | 0.02 | 1 | 11 | 6 | 4 | 2 | 24 | 5 | |
| 1 | 1 | 2 | 1 | 0 | 0 | 0 | 0 | 0 | 0 | 0 | No |
| | | | | | | | | | | | inclusions |
| | | | | | | | | | | | seen |
| | 2 | 0.8 | 0.4 | 0 | 0 | 0 | 0 | 0 | 0 | 0 | No |
| | | | | | | | | | | | inclusions |
| | | | | | | | | | | | seen |
| | 3 | 0.04 | 0.02 | 0 | 0 | 0 | 0 | 0 | 0 | 0 | 6 inclusions |
| | | | | | | | | | | | seen on |
| | | | | | | | | | | | entire |
| | | | | | | | | | | | coverslip |
| 3 | 1 | 2 | 1 | 0 | 0 | 0 | 0 | 0 | 0 | 0 | 15 |
| | | | | | | | | | | | inclusions |
| | | | | | | | | | | | seen on |
| | | | | | | | | | | | entire |
| | | | | | | | | | | | coverslip |
| | 2 | 0.8 | 0.4 | 4 | 7 | 5 | 8 | 4 | 28 | 6 | |
| | 3 | 0.04 | 0.02 | 12 | 24 | 9 | 29 | 22 | 96 | 19 | |
| 2 | 1 | 2 | 1 | 0 | 0 | 0 | 0 | 0 | 0 | 0 | 6 inclusions |
| | | | | | | | | | | | seen on |
| | | | | | | | | | | | entire |
| | | | | | | | | | | | coverslip |
| | 2 | 0.8 | 0.4 | 1 | 2 | 2 | 1 | 5 | 11 | 2 | |
| | 3 | 0.04 | 0.02 | 3 | 2 | 6 | 5 | 7 | 23 | 5 | |

Table 1. Effect of Transient Treatment with maleimides on Chlamydia Infection of McCoy Cell In Vitro. Chlamydia was pre-treated with each of the indicated maleimides for 5 minutes and the compound removed by centrifugation. McCoy cells were then exposed to the compound pre-treated chlamydia for 68 hours and inclusions counted following staining of cells with Lugols iodine.

Anti-Viral Assay

Results

An assessment of viral cytopathic effect was made microscopically on days 5 and 6, but the end point of the experiment was on day 6 when the level of virus infection (as determined by cell viability) was quantified by the use of XTT. There is a good correlation between the microscopic scores for syncytium formation and the OD values obtained by XTT on day 6 and the presence of 10% DMSO in the medium used to treat the virus did not appear to make any difference to the results.

The results of this experiment indicate that the most potent virucidal compound of the series was compound, 1 (IC₅₀=1.0 mM), followed in order of activity by 2 (almost 50% inhibition at 1 mM), 4 (IC₅₀=1.9mM) and 3 which showed no virucidal activity at concentrations up to 1.0 mM.

TABLE 2

| | | Syncytia score day 5 | | | | Syncytia score day 6 | | | |
|---|---|---|---|---|---|---|---|---|---|
| Compound | Conc (mM) | (quadruplicate wells) | | | | (quadruplicate wells) | | | |
| 1 | 1 | ++ | ++ | 0 | 0 | ++ | ++/+++ | 0 | 0 |
| | 0.3 | +++ | +++ | +++ | +++ | +++ | +++ | +++ | +++ |
| | 0.1 | ++++ | ++++ | ++++ | ++++ | ++++ | ++++ | ++++ | ++++ |
| | 0.03 | ++++ | ++++ | ++++ | ++++ | ++++ | ++++ | ++++ | ++++ |
| | 0.01 | ++++ | ++++ | ++++ | ++++ | ++++ | ++++ | ++++ | ++++ |
| | 0.003 | ++++ | ++++ | ++++ | ++++ | ++++ | ++++ | ++++ | ++++ |
| 2 | 1 | +++ | ++ | ++ | 0 | +++ | +++ | ++ | 0 |
| | 0.3 | +++ | +++ | +++ | +++ | ++++ | ++++ | ++++ | ++++ |
| | 0.1 | ++++ | ++++ | ++++ | ++++ | ++++ | ++++ | ++++ | ++++ |
| | 0.03 | ++++ | ++++ | ++++ | ++++ | ++++ | ++++ | ++++ | ++++ |
| | 0.01 | ++++ | ++++ | ++++ | ++++ | ++++ | ++++ | ++++ | ++++ |
| | 0.003 | ++++ | ++++ | ++++ | ++++ | ++++ | ++++ | ++++ | ++++ |
| 3 | 1 | +++ | +++ | +++ | +++ | +++ | +++ | +++ | +++ |
| | 0.3 | +++ | +++ | +++ | +++ | ++++ | ++++ | ++++ | ++++ |
| | 0.1 | +++ | +++ | +++ | +++ | ++++ | ++++ | ++++ | ++++ |
| | 0.03 | +++ | +++ | +++ | +++ | ++++ | ++++ | ++++ | ++++ |
| | 0.01 | +++ | +++ | +++ | +++ | ++++ | ++++ | ++++ | ++++ |
| | 0.003 | ++++ | ++++ | ++++ | ++++ | ++++ | ++++ | ++++ | ++++ |
| 4 | 3 | 0 | 0 | 0 | 0 | 0 | 0 | 0 | +/− |
| | 1 | +++ | +++ | +++ | +++ | ++ | ++ | +++ | +++ |
| | 0.3 | ++++ | ++++ | ++++ | ++++ | ++++ | ++++ | ++++ | ++++ |
| | 0.1 | ++++ | ++++ | ++++ | ++++ | ++++ | ++++ | ++++ | ++++ |
| | 0.03 | ++++ | ++++ | ++++ | ++++ | ++++ | ++++ | ++++ | ++++ |
| | 0.01 | ++++ | ++++ | ++++ | ++++ | ++++ | ++++ | ++++ | ++++ |
| Virus Control + | | ++++ | ++++ | ++++ | ++++ | ++++ | ++++ | ++++ | ++++ |
| DMSO | | | | | | | | | |
| Virus Control + | | ++++ | ++++ | ++++ | ++++ | ++++ | ++++ | ++++ | ++++ |
| Medium only | | | | | | | | | |
| No virus Control | | 0 | 0 | 0 | 0 | 0 | 0 | 0 | 0 |

Key:
0 = No syncytia;
+= <10% syncytia;
++ = 10-25% syncytia;
+++ = 25-50% syncytia;
++++ = 50-100% syncytia.

TABLE 3
Mean Optical Readings at 450 nm

| Conc mM | 4 | 1 | 2 | 3 |
|---|---|---|---|---|
| 3 | 1.6305 | nd | nd | nd |
| 1 | 0.5275 | 1.18075 | 0.9895 | 0.52675 |
| 0.3 | 0.502 | 0.58525 | 0.50925 | 0.50925 |
| 0.1 | 0.50475 | 0.53475 | 0.49825 | 0.51175 |
| 0.03 | 0.4915 | 0.51425 | 0.49275 | 0.50125 |
| 0.01 | 0.48 | 0.50275 | 0.4895 | 0.5085 |
| 0.003 | | 0.4875 | 0.48825 | 0.50175 |

Controls

| | Virus | | |
|---|---|---|---|
| Virus | Control + | | |
| Control | 10% DMSO | No Virus | 50% Control |
| 0.5091 | 0.511 | 1.84955 | 1.1798 |

nd = not determined

TABLE 4

| Compound | IC₅₀ mM |
|---|---|
| 1 | 1.0 |
| 2 | ˜1.0 |
| 4 | 1.9 |
| 3 | >1.0 |

The foregoing description details specific compounds, compositions, methods and uses which can be employed to practice the present invention. However, those skilled in the art will know how to use alternative reliable methods for aiming at alternative embodiments of the invention which are herein encompassed.

1-14. (canceled)

15. A method for the treatment or prophylaxis of a microbial infection of the reproductive tract, or a sexually transmitted infection, comprising administering a poly-maleimide compound to a subject in need of said treatment or prophylaxis.

16. The method of claim 15, wherein the microbial infection is a bacterial, a fungal or a protozoal infection.

17. The method of claim 15, wherein the microbial infection is a viral infection.

18. The method of claim 17, wherein the viral infection is caused by a retrovirus.

19. The method of claim 18, wherein the retrovirus is HIV.

20. The method of claim 15, wherein the poly-maleimide compound is a compound of general formula (I): 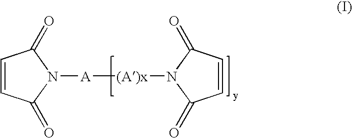
wherein
A is represented by (R¹)ₙ—(R²)ₚ—(R³)_{q}, wherein each of R¹, R² and R³ is independently selected from:
an acyclic aliphatic group having from 1-20 carbon atoms;
a polyalkylene glycol group having an average molecular weight in the range 100 to 1000;
a cyclic aliphatic group having from 4-20 carbon atoms;
a monocyclic or polycyclic aromatic hydrocarbon group having from 6-18 ring carbon atoms;
a non-aromatic heterocyclic group having from 3 to 8 ring atoms, or
a monocyclic or polycyclic aromatic heterocyclic group having from 5 to 20 ring atoms;
and n, p and q each represent 0 or 1 such that the sum of n+p+q=1-3;
or A substituted by one or more groups independently selected from: 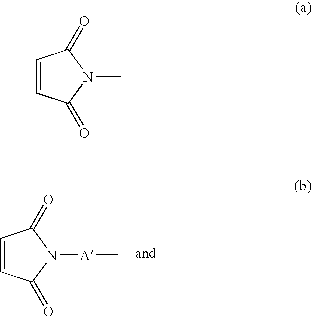
wherein A′, which may be the same as or different from A, represents a group (R¹)ₙ—(R²)ₚ—(R³)_{q}, wherein R¹, R², R³, n, p and q are as defined above and wherein X is
0 or 1 and y is 1 to 6;
or a salt, ester or other physiologically equivalent derivative thereof.

21. The method of claim 20, wherein the poly-maleimide compound is a compound of formula (II): 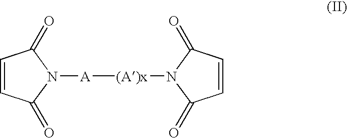
wherein A, A¹ and x are as defined in claim 20;
or a salt, ester or other physiologically equivalent derivative thereof.

22. A pharmaceutical formulation, comprising a poly-maleimide compound of general formula (I): 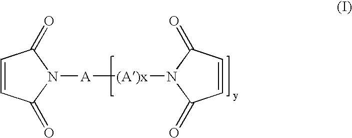
wherein
A is represented by (R¹)ₙ—(R²)ₚ—(R³)_{q} wherein each of R¹, R² and R³ is independently selected from:
an acyclic aliphatic group having from 1-20 carbon atoms;
a polyalkylene glycol group having an average molecular weight in the range 100 to 1000;
a cyclic aliphatic group having from 4-20 carbon atoms;
a monocyclic or polycyclic aromatic hydrocarbon group having from 6-18 ring carbon atoms;
a non-aromatic heterocyclic group having, from 3 to 8 ring atoms, or
a monocyclic or polycyclic aromatic heterocyclic group having from 5 to 20 ring atoms;
and n, p and q each represent 0 or 1 such that the sum of n+p+q=1-3;
or A substituted by one or more groups independently selected from: 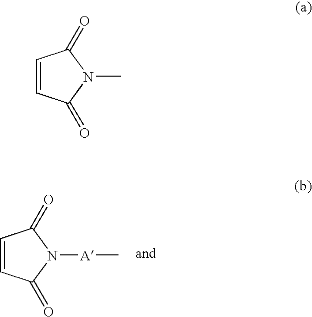
wherein A′, which may be the same as or different from A, represents a group (R¹)ₙ—(R²)ₚ—(R³)_{q}, wherein R¹ , R² , R³ , n, p and q are as defined above and wherein X is 0 or 1 and y is 1 to 6;
or a salt, ester or other physiologically equivalent derivative thereof.

23. The pharmaceutical formulation of claim 22, wherein the poly-maleimide compound is a compound of formula (II): 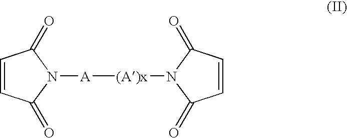
wherein A, A¹ and x are as defined in claim 22;
or a salt, ester or other physiologically equivalent derivative thereof.

24. A compound of formula (V) 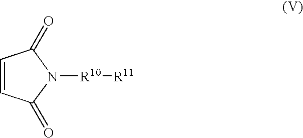
wherein R¹¹ is 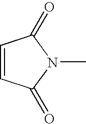
and R¹⁰ is C₂₋₁₀ alkyl, or a group R²⁰—R²¹—R²²
wherein R²⁰ is aryl;
R²¹ is —(OCH₂CH₂)ₙ— wherein n is 3 to 15;
R²² is a group O—R²³ wherein R²³ is aryl or C₁₋₁₀ alkyl.

25. The compound of claim 24 selected from any one of: [chemical structure]
